(19) United States
(12) Patent Application Publication
(10) Pub. No.: US 2011/0020494 A1
(43) Pub. Date: Jan. 27, 2011

(54) GROWTH MEDIUM FOR LACTIC ACID BACTERIA

(76) Inventor: Lars Petersen, Muskego, WI (US)

Correspondence Address: STEPTOE & JOHNSON LLP, 1330 CONNECTICUT AVENUE, N.W., WASHINGTON, DC, 20036, US

(21) Appl. No.: 12/680,195

(22) PCT Filed: Oct. 30, 2007
(86) PCT No.: PCT/IB2008/003302

§ 371 Date: Oct. 7, 2010

Related U.S. Application Data
(60) Provisional application No. 60/983,909, filed on Oct. 30, 2007.

(51) Int. Cl. C12N 1/20; A23C 19/00; A23C 9/123

(52) U.S. Cl. 426/36; 435/252.1; 435/252.9; 435/253.4; 426/43; 426/580

(57) ABSTRACT

The present invention provides lactic acid bacterial cultures. In some particularly preferred embodiments, the present invention provides cultures comprising lactic acid bacteria, a dairy substrate (e.g., milk or cream), at least one formate source, and at least one purine source. In some further embodiments, the cultures comprise exogenous lactase enzyme. The present invention also provides cultures of cells comprising the culture medium and lactic acid bacteria. The present invention further provides starter compositions (i.e., starter cultures) for starting the culture. In some embodiments, the compositions of the present invention find use in the production of fermented milk products (e.g., yogurt, cheese and/or fermented milk beverages).

The present application claims priority to U.S. Prov. Patent Appln. Ser. No. 60/983,909, filed Oct. 30, 2007, and U.S. Prov. Patent Appln. Ser. No. 61/020,205, filed Jan. 10, 2008.

### FIELD OF THE INVENTION

The present invention provides lactic acid bacterial cultures. In some particularly preferred embodiments, the present invention provides cultures comprising lactic acid bacteria, a dairy substrate (e.g., milk or cream), at least one formate source, and at least one purine source. In some further embodiments, the cultures comprise exogenous lactase enzyme. The present invention also provides cultures of cells comprising the culture medium and lactic acid bacteria. The present invention further provides starter compositions (i.e., starter cultures) for starting the culture. In some embodiments, the compositions of the present invention find use in the production of fermented milk products (e.g., yogurt, cheese and/or fermented milk beverages).

### BACKGROUND OF THE INVENTION

Fermented milk products include dairy foods that have been fermented using lactic acid bacteria such as Lactobacillus and Lactococcus. The fermentation process converts carbohydrate into lactic acid, which increases the shelf-life of the product, as well as enhancing the taste and improves the digestibility of milk. Cheese, yogurt, sour cream and crème fraiche are examples of such products.

### SUMMARY OF THE INVENTION

The present invention provides lactic acid bacterial cultures. In some particularly preferred embodiments, the present invention provides cultures comprising lactic acid bacteria, a dairy substrate (e.g., milk or cream), at least one formate source, and at least one purine source. In some further preferred embodiments, the cultures comprise exogenous lactase enzyme. The present invention also provides cultures of cells comprising the culture medium and lactic acid bacteria. The present invention further provides starter compositions (i.e., starter cultures). In some embodiments, the compositions of the present invention find use in the production of fermented milk products (e.g., yogurt, cheese and/or fermented milk beverages).

In some embodiments, the invention provides a bacterial culture comprising a dairy substrate, a population of lactic acid bacteria, lactase, a source of formate, and a source of purine. In some further embodiments, the cultures further comprise at least one exogenous lactase enzyme. In some preferred embodiments, the bacterial culture comprises a dairy substrate, a population of lactic acid bacteria, an exogenous lactase, a source of formate, and a source of purine. The present invention also provides cultures of cells comprising the culture medium and lactic acid bacteria. The present invention further provides starter compositions (i.e., starter cultures). In some embodiments, the compositions of the present invention find use in the production of fermented milk products (e.g., yogurt, cheese and/or fermented milk beverages).

In some embodiments, the present invention provides compositions for starting a culture (i.e., a “starter culture”). In some embodiments, the composition is frozen and comprises a population of lactic acid bacteria and exogenous lactase enzyme. In some preferred embodiments, the composition is used in methods that include combining the above-described starter composition with a dairy substrate (e.g., milk or cream) to make a bacterial culture. In some embodiments, the bacterial culture is maintained under culture conditions to make a fermented milk product (e.g., yogurt, cheese or fermented milk beverages).

The present invention also provides bacterial cultures. In some embodiments, the bacterial culture comprises: a dairy substrate; a population of lactic acid bacteria; and exogenous lactase enzyme. In some embodiments, the dairy substrate is milk or cream. It is contemplated that milk from any suitable animal will find use in the present invention, including but not limited to cow, goat, sheep, yak, buffalo, bison, etc. Indeed it is intended that the present invention encompass milk obtained from any mammal. In some preferred embodiments, the bacteria are members of the genera Lactococcus, Lactobacillus or Streptococcus. In some preferred embodiments, the dairy substrate is not a lactose-hydrolyzed dairy substrate. In some of these embodiments, the lactase enzyme is present at a concentration of about 0.1 μg/ml to about 10 mg/ml. In some further embodiments, the lactic acid bacteria population is in lag phase in this culture. In some alternative embodiments, the culture is prepared by inoculating milk with a starter composition comprising the lactase enzyme and lactic acid bacteria. In some further alternative embodiments, the bacterial culture is prepared using a method that includes combining a dairy substrate with a population of lactic acid bacteria and isolated lactase enzyme. In some embodiments, the population of lactic acid bacteria and the isolated lactase enzyme are combined simultaneously, while in other embodiments the lactic acid bacteria and isolated lactase enzyme are sequentially combined. In some further embodiments, the bacterial cultures further comprise at least one source of formate. In some additional embodiments, the bacterial cultures are frozen and/or freeze-dried.

The present invention also provides further compositions for starting cultures. In some embodiments, the composition is frozen and/or freeze-dried, and comprises: a population of lactic acid bacteria; at least one formate source; and at least one purine source. In some embodiments, the formate source is a formate salt (e.g., sodium formate). In some embodiments, the purine source is inositol 5′ monophosphate, inosine, hypoxantine, guanine, or disodium-5′-inosinate. However, it is not intended that the present invention be limited to these specific formate and/or purine sources, as any suitable source finds use in the present invention. In some embodiments, the starter composition comprises a population of lactic acid bacteria at a concentration in the range of about 10⁶ to about 10¹³ CFU/g, a source of formate at a concentration of about 10% to about 50% (w/w); and a source of purine at a concentration of about 10% to about 50% (w/w).

The present invention provides additional methods. In some embodiments, the methods include combining the above-described starter composition with a dairy substrate (e.g., milk or cream) to make a bacterial culture. In some embodiments, the bacterial culture is maintained under culture conditions to make a fermented milk product (e.g., yogurt, cheese, or fermented milk beverages).

The present invention also provides further bacterial cultures. In some embodiments, the bacterial culture comprises: a dairy substrate; a population of lactic acid bacteria; a source of formate; and a source of purine. In some embodiments, the dairy substrate is milk or cream. It is contemplated that milk from any suitable animal will find use in the present invention, including but not limited to cow, goat, sheep, yak, buffalo, bison, etc. Indeed it is intended that the present invention encompass milk obtained from any mammal. In some preferred embodiments, the bacteria are members of the genera Lactococcus, Lactobacillus or Streptococcus. In some of these embodiments, the formate source is present at a concentration of about 0.25 ppm to about 50 ppm, and the source of purine is present at a concentration of about 0.5 ppm to about 100 ppm. In some embodiments, the lactic acid bacteria population is in lag phase in this culture. In some embodiments, the culture is prepared by inoculating milk with a starter composition comprising sources of formate and purine, and lactic acid bacteria.

It is contemplated that any of the above-summarized lactase-related embodiments will find use when employed independently of or in combination with any of the formate/purine-related embodiments also summarized. For example, in some embodiments, the composition for starting a culture comprises: a population of lactic acid bacteria, exogenous lactase enzyme, a source of formate; and a source of purine. In some other embodiments, the bacterial culture comprises: a dairy substrate, a population of lactic acid bacteria, exogenous lactase enzyme, a source of formate; and a source of purine. In some embodiments, cultures are made by combining a dairy substrate with a starter composition containing all of the added components. In some embodiments, the dairy substrate is simultaneously combined with the added components, while in other embodiments, the dairy substrate is sequentially combined with the added components.

It is contemplated that any of the above-described bacterial cultures will find use in methods comprising: maintaining the bacterial culture under conditions suitable for fermentation, to make a fermented milk product. In some embodiments, the methods provide for the production of lactic acid by the population of lactic acid bacteria, and lowering of the pH of the culture. In some embodiments, the fermented milk product is yogurt, cheese or a fermented milk beverages. In some further embodiments, the methods further include adding a flavoring and/or a stabilizer to the fermented milk product. In yet additional embodiments, the methods further include packaging the fermented milk product. The present invention also provides fermented products made using any of the methods provided herein.

### DESCRIPTION OF THE FIGURES

FIG. 1 provides a graph of pH relative to time, for four cultures of lactic acid bacteria.

### DESCRIPTION OF THE INVENTION

The present invention provides lactic acid bacterial cultures. In some particularly preferred embodiments, the present invention provides cultures comprising lactic acid bacteria, a dairy substrate (e.g., milk or cream), at least one formate source, and at least one purine source. In some further embodiments, the cultures comprise exogenous lactase enzyme. In some further embodiments, the cultures comprise exogenous lactase enzyme combined with at least one formate source, and/or at least one purine source. The present invention also provides cultures of cells comprising the culture medium and lactic acid bacteria. The present invention further provides starter compositions (i.e., starter cultures) for starting the culture. In some preferred embodiments, the starter compositions comprise exogenous lactase enzyme which can be combined with at least one formate source, and/or at least one purine source. In some embodiments, the compositions of the present invention find use in the production of fermented milk products (e.g., yogurt, cheese and/or fermented milk beverages).

As described further herein, in some embodiments, the present invention provides bacterial cultures comprising a dairy substrate (e.g., milk), a population of lactic acid bacteria, and exogenous lactase enzyme. Other embodiments provide a bacterial culture containing a dairy substrate, a population of lactic acid bacteria, at least one formate source, and at least one purine source. In some embodiments, these bacterial cultures are starter compositions comprising a population of lactic acid bacteria, at least one source of formate, and at least one source of purine. In some preferred embodiments, the starter composition is used to inoculate a dairy substrate, in order to produce a culture. Indeed, the present invention provides a variety of compositions, including bacterial cultures and compositions for starting such cultures. The present invention also provide various methods for using the present compositions.

In some further embodiments, the lactase-related embodiments and the format/purine-related embodiments are combined (e.g., providing a bacterial culture containing a dairy substrate, a population of lactic acid bacteria, exogenous lactase enzyme, at least one formate source, and at least one purine source). In some further embodiments at least one formate source or at least one purine source find use.

Although it is not intended that the present invention be limited to any particular mechanism of action nor theory, it is contemplated that the added lactase enzyme, and/or sources of formate and purine, facilitate the growth of the lactic acid bacteria in the culture, particularly during the early growth stages (i.e., when the bacteria are in lag phase).

In some preferred embodiments, the added components allow the culture to reach a steady state pH in the range of about 3.6 to about 6.0, earlier than an equivalent culture that does not contain the added lactase enzyme, and/or formate and/or purine sources. Depending on the culture conditions used, cultures reach a steady state pH in less than about 70%, less than about 80%, less than about 90% or less than about 95% of the time of an equivalent culture that does not contain the added components. In some embodiments, the resultant time saving is in the region of about 0.5 to about 10 hours per fermentation run. In some further embodiments, the time saving is from about 1 to about 2 hours per fermentation run.

In some embodiments, it is contemplated that the sources of formate and purine in the culture medium negate the inhibitory effects of oxygen in the culture medium. In some lactase-related embodiments, the resultant fermented milk product tastes sweeter, and contain less lactose than fermented milk products produced without added lactase. However, it is not intended that the present invention be limited to any particular mechanism of action, nor theory.

### DEFINITIONS

Unless defined otherwise herein, all technical and scientific terms used herein have the same meaning as commonly understood by one of ordinary skill in the art to which this invention pertains. Although any methods and materials similar or equivalent to those described herein find use in the practice or testing of the present invention, the preferred methods and materials are provided herein. Unless otherwise indicated, nucleic acids are written left to right in 5′ to 3′ orientation; amino acid sequences are written left to right in amino to carboxy orientation, respectively.

All patents and publications, including all sequences disclosed within such patents and publications, referred to herein are expressly incorporated by reference. Unless defined otherwise herein, all technical and scientific terms used herein have the same meaning as commonly understood by one of ordinary skill in the art to which this invention pertains. Although any methods and materials similar or equivalent to those described herein find use in the practice of the present invention, preferred methods and materials are described herein. Accordingly, the terms defined immediately below are more fully described by reference to the Specification as a whole. Also, as used herein, the singular terms “a,” “an,” and “the” include the plural reference unless the context clearly indicates otherwise. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “food” refers to any nutritional item that provides nourishment to a plant and/or animal. It is not intended that the term be limited to any particular item, as it is used in reference to any substance taken into and assimilated by a plant or animal to keep it alive. It is also not intended that the term be limited to “solid” food, as liquid nourishment is encompassed by the definition. Indeed in some embodiments, liquid nourishment is preferred over solid food items. In some preferred embodiments, the term is specifically used in reference to food for human consumption.

As used herein, the term “feed” refers to any nutritional item that provides nourishment to non-human animals. It is not intended that the term be limited to any particular item, as it is used in reference to any substance taken into and assimilated by a plant or animal to keep it alive. It is also not intended that the term be limited to “solid” food, as liquid nourishment is encompassed by the definition. Indeed in some embodiments, liquid nourishment is preferred over solid food items.

As used herein, the term “isolated” with respect to bacteria, refers to bacteria that have been isolated from their natural environment and cultured in vitro prior to use.

With respect to a protein or other molecule, the term “isolated” refers to a protein or other molecule that is removed from at least one component with which it is naturally associated.

As used herein, the term “purified” refers to a molecule that it is at least about 50%, at least about 90%, or at least about 99% free of components with which it is naturally associated.

As used herein, the term “dairy substrate” refers to milk of an animal, as well as components of the milk (e.g., cream), and combinations of milk and cream (e.g., half and half).

As used herein, the term “milk” refers to whole milk produced by an animal, as well as processed forms thereof. Indeed, any suitable form of milk finds use in the present invention, including milk that includes or does not include whey, as well as whole milk, raw milk, skim milk, evaporated milk, reconstituted milk, condensed milk, pasteurized milk, unpasteurized, homogenized, non-homogenized, or re-hydrated milk powder. Indeed, it is contemplated that milk in any form will find use in the present invention. In some embodiments, cream obtained from any suitable milk finds use.

It is intended that the term encompass milk from any suitable animal. Thus, milk obtained from any suitable mammal finds use in the present invention, including but not limited to milk obtained from sources such as bovines, ovines, caprines, porcines, yaks, buffaloes, water buffaloes, bison, canines, felines, primates, equines, etc.

As used herein, the term “cream” refers to the higher-butterfat layer that is skimmed from the top of milk before homogenization. In non-homogenized milk, the fat contained in the milk is lighter than the other milk components and rises to the top of the milk. It is intended that any form of cream be encompassed, including but not limited to whole cream, light cream, coffee cream, table cream, medium cream, whipping or light whipping cream, extra heavy or manufacturer's cream, clotted cream, double cream, etc. In some embodiments, the cream is rehydrated cream powder. Indeed, it is not intended that the present invention be limited to any particular form of cream. It is also intended that as with milk, any suitable source of cream finds use in the present invention.

As used herein, the term “lactose-hydrolyzed dairy substrate” refers to a dairy substrate that has been pre-treated with lactase enzyme to reduce lactose levels. Thus, these dairy substrates have lower lactose levels than a non-lactose-hydrolyzed dairy substrate.

As used herein, the term “lactic acid bacteria” refers to any of a clade of Gram-positive, acid tolerant, non-sporulating, non-respiring rod or cocci that produce lactic acid as the major metabolic end-product of carbohydrate fermentation. Such bacteria include, but are not limited to, bacteria of the genus Lactococcus, Lactobacillus and Streptococcus.

As used herein, the term “culture” refers to any sample or item that contains one or more microorganisms. “Pure cultures” are cultures in which the organisms present are only of one strain of a particular genus and species. This is in contrast to “mixed cultures,” which are cultures in which more than one genus and/or species of microorganism are present. In some embodiments of the present invention, pure cultures find use.

As used herein, the term “Lactobacillus” refers to members of the genus Lactobacillus, in the family Lactobacillaceae. These bacteria are Gram-positive facultatively anaerobic bacteria that represent a major part of the bacterial group often referred to as “lactic acid bacteria.” Various species of Lactobacillus have been identified, including but not limited to L. acidophilus, L. bulgaricus, L. casei, L. delbrueckii, L. fermentum, L. plantarum, L. reuteri, etc. It is intended that the genus include species that have been reclassified (e.g., due to changes in the speciation of organisms as the result of genetic and other investigations) or renamed for marketing and/or other purposes.

As used herein, the terms “microbiological media,” “culture media,” and “media” refer to any suitable substrate for the growth and reproduction of microorganisms. The term encompasses solid plated media, as well as semi-solid and liquid microbial growth systems.

As used herein, the term “additive” refers to any compound, composition, element and/or other material that is added to the culture media to enhance microbial growth and/or production of a desired protein.

As used herein, the term “lactase” refers to a glycoside hydrolase that catalyzes the hydrolysis of the disaccharide lactose into constituent galactose and glucose monomers. Lactases have an activity described as EC 3.2.1.108, according to IUBMB enzyme nomenclature. The systematic name for a lactase is β-D-galactopyranosyl-(1→4)-α-D-glucopyranose.

As used herein, the term “lactase unit” refers to the measurement of lactase activity. For acid lactases, one lactase unit hydrolyzes 1 uM of o-nitrophenyl-beta-D-galactoside to o-nitrophenol and beta-galactosidase per minute at pH 4.5, and 30° C. For neutral lactases, the unit is the same, but the pH is 6.5 and the temperature is 37° C.

As used herein, the term “exogenous lactase enzyme,” when used with reference to a bacterial culture that contains such an enzyme, refers to an enzyme that is not secreted by the bacteria in the culture (i.e., the lactase is added to the medium).

As used herein, the term “endogenous lactase enzyme” refers to lactase that is produced by the lactic acid bacteria in a culture. In some embodiments, the endogenous lactase enzyme is over-expressed by the lactic acid bacteria, such that the concentration of lactase in the composition is greater than it would be if the lactic acid bacteria did not overexpress the lactase enzyme.

As used herein, the term “frozen” refers to a solid state composition that is at a low temperature (e.g., less than about 0° C.). In some preferred embodiments, the term refers to a composition that is at a temperature of about −20° C. or less.

As used herein, the terms “freeze dried” and “lyophilized” refer to a solid state composition that is dried by freezing the composition in a high vacuum. As with freezing, these methods are well known in the art and any suitable method finds use in the present invention.

As used herein, the terms “source of formate” and “formate source” refer to a compound that when added to a culture of cells, provides formate. In some embodiments, the source of formate releases formate into a growth medium, while in other embodiments, the formate source is metabolized to produce formate. In some preferred embodiments, the formate source is exogenous. In some particularly preferred embodiments, formate is not provided by the dairy substrate.

As used herein, the terms “source of purine” and “purine source” refer to a compound that when added to a culture of cells, provides purine nucleotides. In some embodiments, the source of purine releases purine into a growth medium, while in other embodiments, the purine source is metabolized to produce purine. In some preferred embodiments, the purine source is exogenous. In some particularly preferred embodiments, purine is not provided by the dairy substrate.

As used herein, the term “exogenously added,” with reference to a growth medium, refers to a compound that is added in addition to other components of the growth medium. For example, if a growth medium contains a dairy substrate and a source of formate that is exogenously added, the source of formate is not present in the dairy substrate. Rather, the source of formate is added in addition to the dairy substrate.

As used herein, the term “fermented milk product” refers to a food product made by fermentation of dairy substrate using at least one species of lactic acid bacteria. In some embodiments, the products have a low pH (e.g., a pH of about 3.6 to about 6.0). In some preferred embodiments, the products contain lactic acid. Fermented milk products are provided in any form, including frozen, liquid or solid form. In some embodiments, the products contain living bacteria, while in some alternative embodiments, no living bacteria are present. Fermented milk products include, but are not limited to cheese, yogurt, sour cream, crème fraiche, and fermented milk beverages.

### Bacterial Cultures

As described herein, the present invention provides bacterial cultures. In some embodiments, the bacterial culture comprises: dairy substrate (e.g., milk or cream); a population of lactic acid bacteria; at least one source of formate; and at least one source of purine. The present invention also provides growth media comprising all of the above components with the exception of the population of lactic acid bacteria.

In some embodiments, the bacterial culture comprises: dairy substrate; a population of lactic acid bacteria; and exogenous lactase enzyme. The present invention also provides growth media comprising all of the above components with the exception of the population of lactic acid bacteria.

In some further embodiments, the bacterial culture comprises a dairy substrate, a population of lactic acid bacteria, an exogenous lactase enzyme, at least one source of formate, and/or at least one source of purine. The present invention also provides growth media comprising all of the above components with the exception of the population of lactic acid bacteria.

In some preferred embodiments, by volume, the major component of the bacterial culture (at the beginning of fermentation) is the dairy substrate (e.g., milk). As indicated herein, the dairy substrate component of the culture is milk from any suitable animal species. In some embodiments, the dairy substrate comprises at about least about 80%, at least about 90%, at least about 95%, at least about 99%, or at least about 99.9% of the volume of the culture. Any suitable form of milk finds use in the present invention, including milk that includes or does not include whey, as well as whole milk, raw milk, skim milk, evaporated milk, reconstituted milk, condensed milk, or re-hydrated milk powder. Indeed, it is contemplated that milk in any form will find use in the present invention.

In some embodiments, the dairy substrate is not lactose-hydrolyzed milk (i.e., it has not been pre-treated to reduce the lactose content of the milk (e.g., by either enzyme treatment or filtration). In some embodiments, the concentration of lactose in a dairy substrate is at least about 4% (e.g., about 4% to about 5% or about 4.5% to about 5%) lactose. In some preferred embodiments, the dairy substrate is cow's milk having a lactose concentration of about 4.7% (w/v), goat's milk having a lactose concentration of about 4.1%, buffalo's milk having a lactose concentration of about 4.86%, yak milk having a lactose concentration of about 4.93%, or sheep's milk having a lactose concentration of about 4.6%. However, it is not intended that the present invention be limited to any particular lactose concentration nor milk from any particular species.

In some embodiments, the population of lactic acid bacteria is a population of lactic acid bacteria that has GRAS status. In some preferred embodiments, the population of lactic acid bacteria is in the Order Lactobacillales. In some embodiments, the bacteria in the culture are a single species (i.e., a “pure culture”), while in other embodiments, the bacteria comprise a mixture of species within the following genera: Abiotrophia, Aerococcus, Bifidobacterium, Carnobacterium, Enterococcus, Lactobacillus (including, but not limited to: L. acidophilus, L. brevis, L. delbrueckii ssp. bulgaricus, L. casei, L. delbrueckii, L. fermentum, L. helveticus, L. plantarum, L. rhamnosus, L. reuteri, and/or L. sanfranciscensis), Lactococcus (including, but not limited to: L. garvieae, L. lactis, L. piscium, L. plantarum, and/or L. raffinolactis), Leuconostoc, Oenococcus, Pediococcus, Streptococcus (including, but not limited to S. thermophilus), Tetragenococcus, Vagococcus and Weissella. The population of bacteria in the culture may be at any phase of growth, including lag phase, logarithmic phase, and stationary phase, or may be dormant.

The exogenous lactase enzyme is any suitable lactase that is active in milk. Such enzymes are known in the art and are commonly employed in treatments for lactose intolerance, and for other applications (e.g., for pre-treatment of milk to lower lactose levels; See e.g., Reprod. Nutr. Dev., 42:127-32 [2002]). In some embodiments, the lactase is a commercially available lactase, (e.g., the lactase of Kluyveromyces fragilis [K. marxianus var. marxianus]; Artolozaga et al. Bioseparation 7:137-43 [1998]), having a pH optimum (pH 6.5-7.0), or a lactase from Aspergillus oryzae or A. niger, having pH optima (pH about 4.5 to about 6.0 and about 3.0 to about 4.0, respectively). Suitable enzymes are well known to those in the art (See e.g., Pivarnik et al., Adv. Food Nutr. Res., 38:1-102 [1995]). In some embodiments, the lactase is an acid lactase having an optimal lactase activity at a pH ranging from approximately 4 to approximately 6. Alternatively, in some other embodiments, the lactase is a neutral lactase having an optimal lactase activity at a pH ranging from approximately 5.5 to approximately 7. In some preferred embodiments, acid lactases are used. In some further preferred embodiments, acid and neutral lactases are used. Non-limiting examples of acid lactase is Lactase DS (Amano Enzyme, Nagoya, Japan). Non-limiting examples of neutral lactase include Maxilact® (DSM, Herleen, The Netherlands) and Lactase Godo (Godo Shusei Co., Tokyo, Japan). In some embodiments, the enzyme is purified, while in other embodiments it is obtained from a natural isolate, or is produced using recombinant means.

The lactase enzyme is present at a concentration that provides the desired effect. In some preferred embodiments, the lactase enzyme is present at a concentration in the range of about 0.1 μg/ml to about 10 mg/ml, although it is contemplated that concentrations outside of this range will find use in the present invention. In some embodiments, the lactase is present at a concentration of about 0.2 μg/ml to about 100 μg/ml. In still further embodiments, the concentration of lactase ranges from 0.5 to 20 units of lactase per milliliter of milk.

In some most particularly preferred embodiments, the lactase present in the culture is exogenous (i.e., it is not secreted by the cells in the culture). In particularly preferred embodiments, the lactase is at a concentration that is significantly higher then the level of any lactase secreted by the bacteria in the culture.

As indicated herein, the formate source(s) comprise(s) any compound that releases formate into solution, or that can be metabolized by a lactic acid bacterium to produce formate. In some embodiments, a formate salt is used (e.g., sodium formate, potassium formate, calcium formate, and ammonium formate). In some preferred embodiments, the formate source is present in the bacterial culture at a concentration of about 0.5 ppm to about 50 ppm, (e.g., about 1 ppm to about 20 ppm, or about 2 ppm to about 10 ppm). In some particularly preferred embodiments, the source of formate is exogenous in that it is not supplied by the dairy substrate.

Any suitable source of purine that releases purine into solution, or that can be metabolized by a lactic acid bacterium to produce a purine nucleotide finds use in the present invention. In some embodiments, the purine source is metabolizable into any purine required for cell growth (e.g., AMP, ADP, ATP, cAMP, NADH and GTP). Suitable purines include, but are not limited to inositol 5′ monophosphate, inosine, hypoxanthine, and guanine. In some embodiments, the source of purine is present in the bacterial culture at a concentration of about 1 ppm to about 100 ppm (e.g., about 2 ppm to about 50 ppm, or about 5 ppm to about 20 ppm). In some particularly preferred embodiments, the source of purine is exogenous in that it is not supplied by the dairy substrate.

The bacterial culture is prepared using any suitable method. Indeed, it is not intended that the present invention be limited to any particular method for producing the bacterial culture. However, in some preferred embodiments and as described in greater detail below, the culture is made by combining a dairy substrate (e.g., milk or cream), with a starter composition that contains lactic acid bacteria and the sources of formate and/or purine. In some further particularly preferred embodiments, the culture further comprises at least one lactase enzyme. These starter compositions are described in greater detail below. In some alternate embodiments, the culture is made by independently adding the various components to the dairy substrate simultaneously, while in other embodiments, the various components are sequentially added to the dairy substrate. In some further embodiments, the culture is prepared by mixing a frozen starter composition comprising the cells and sources of purine and formate, and a powdered or liquid form of the enzyme with the dairy substrate. In some embodiments, the starter composition and the enzyme are added simultaneously, while in some alternative embodiments they are added in rapid succession (i.e., within 5 minutes of the first addition).

### Fermentation Methods

The above-described bacterial culture finds use in methods that include maintaining the culture under conditions suitable for fermentation. In some particularly preferred embodiments, the bacterial culture is used to produce a fermented milk product. In some embodiments, the conditions are suitable for the production of lactic acid by the population of lactic acid bacteria, and/or for lowering the pH of the culture (e.g., to a pH of about 3.6 to about 6.0).

In some embodiments, the fermented milk product is cheese, while in other embodiments, it is yogurt, cultured sour cream, kefir, buttermilk, acidophilus milk, crème fraîche, kumis, shubat, quark, filmjölk, Smetana, skyr, villik, or koumiss. In some embodiments, the yogurt is thermophilic fermented milk made using Lactobacillus bulgaricus or Streptococcus thermophilus). In some embodiments, the cultured sour cream is a mesophilic fermented pasteurized cream with an acidity of at least about 0.5%, made using Lactococcus lactis subsp. lactis). In some further embodiments, the cultured buttermilk is a mesophilic fermented pasteurized milk made using Lactococcus lactis (e.g., Lactococcus lactis subsp. lactis, Lactococcus lactis subsp. cremoris, Lactococcus lactis biovar. diacetylactis) and/or Leuconostoc (e.g., Leuconostoc mesenteroides subsp. cremoris). In some other embodiments, the acidophilus milk is a thermophilic fermented milk beverage, often low-fat (i.e., about 2%, or about 1.5%) or nonfat (i.e., about 0.5%), made using, Lactobacillus acidophilus. In some further embodiments, the crème fraiche is a mesophilic fermented cream made using lactic acid bacteria that are naturally presenting in the cream. In some still additional embodiments, the product is kumis made using mare milk, shubat made using camel milk, quark, filmjölk, smetana, skyr or viili, or koumiss. Methods for making such fermented milk products are well known, and are readily adapted by the addition of sources of purine and formate, as described above.

In some preferred embodiments, a flavoring composition is added to the culture prior to use. In some embodiments, the flavoring composition is savory and in some embodiments contains salt, spices, herbs, flavorings, colorants, purees or pieces of vegetables and/or cereals. In some alternative embodiments, the flavoring composition is sweet and in some embodiments, contains sugar, honey, syrup, flavorings, colorants, compote, jam, marmalade, preserves, pulps, and/or pieces of fruit (e.g., dried, preserved, candied, and/or fresh).

In some embodiments, the flavoring composition is stabilized with a stabilizer, such that is given the necessary consistency for extrusion. Suitable stabilizers include but are not limited to food gums (e.g., locust bean gum, guar gum, xanthan gum, starches, short texture native or modified starch, and pectin). In some embodiments, the food gums are used individually, while in other embodiments, they are used as an admixture. In some embodiments, the methods of the present invention further include packaging the fermented milk product (e.g., to make a product that can be shipped and commercially sold). In other embodiments, the fermented milk product is shipped to a remote location prior to packaging. In yet other embodiments, the fermented milk product is old as a drink or food (e.g., a cheese, fermented milk drink, lactic acid bacteria beverage, yogurt, cultured milk, kefir, or the like). As indicated above, in some embodiments, the product is plain, while in other embodiments, it is flavored, sweetened or contains additional components (e.g., fruit). The product is provided in any suitable form (e.g., soft, hard, liquid, or a frozen product).

The present invention also provides fermented products made using the methods provided herein. In some preferred embodiments, the fermented milk product tastes sweeter than equivalent products made without the additional components. The relative sweetness of lactose is approximately 20% of sucrose whereas glucose and galactose are 60% as sweet as sucrose (Fennema, 1996; Iversen, 1983). Hydrolysis of 70% of lactose present in milk increases the sweetness by an amount comparable to the addition of approximately 2% of sucrose (Lindamood et al. 1989). The sweeter taste enables dairies and/or dairy processors to decrease the level of added natural or artificial sugars to the fermented products. This results in healthier fermented products since these products contain a reduced amount of sweetener(s). It is contemplated that the present invention will provide cost savings for the dairy industry, as a decreased amount of added sweetener in fermented products is facilitated by the present invention. It is also contemplated that the present invention will provide cost savings to the agro food industries (e,g, the bakery industry), as fermented products having a sweeter taste to make food product (e.g., cakes, pancakes, biscuits) with a reduced amount of added sweetener are provided. In some embodiments, this additional sweetness is at least partially due to the production of galactose and glucose (i.e., from lactose metabolism). In some further embodiments, the fermented products have a more appealing texture when frozen due to reduced crystallization than similar products made using standard methods known in the art. In some embodiments, the fermented product is a “low lactose” product, which contains less lactose (e.g., at least about 10% less, at least about 20% less, at least about 30% less, at least about 50% less, or at least about 70% less) than products made without the additional components. In some embodiments, the final lactose concentration of a product made using the methods of the present invention is about 0.1% to about 3%, while in some preferred embodiments the concentration is from about 0.5% to about 2%. In some embodiments, the lactose level is reduced to a level below 100 ppm in the final fermented product, or to a level that permits the fermented product to be considered or labeled as “lactose free.” These embodiments enable the dairy industry to have a natural way of reducing lactose in fermented product. This also provides benefits for those who are lactose intolerant, as these individuals may be able to enjoy dairy products made using the compositions and methods of the present invention.

### Starter Compositions

As indicated above, the present invention provides starter culture compositions. In some embodiments, the starter composition is combined with a dairy substrate (e.g., milk or cream), to produce a cell culture, as described in greater detail herein. In some embodiments, the starter composition is a frozen composition. In some particularly preferred embodiments, the starter culture is packaged in a way that facilitates easy addition of the composition to a dairy substrate, in order to produce a culture. In some embodiments, the present invention provides starter compositions comprising frozen pellets of cell culture, at least one source of formate and/or at least one source of purine.

In some embodiments, the starter composition comprises lactic acid bacteria, and exogenous lactase enzyme at a concentration higher than its concentration in a cell culture without the addition of exogenous lactase. In some other embodiments, the starter composition contains lactic acid bacteria, a source of formate, and/or a source of purine, wherein the concentrations of formate and/or purine are higher than their concentrations in the culture of cells without the added sources of formate and/or purine. In some embodiments, the starter composition contains frozen pellets of cell culture, a source of formate, and a source of purine. As noted above, in some embodiments, a starter composition comprises the cells, the sources of purine and formate, and exogenous lactase.

Starter compositions containing lactic acid bacteria are well known in the art (See e.g., U.S. Patent Appln. Publns. 20060204484, 20050053582 and 20050032196, as well as Speckman et al. J. Dairy Sci., 57:165-173 [1974]; Jakubowska et al., Acta Microbiol. Pol., 1980 29:135-44 [1980]); Gibson et al., Appl. Microbiol., 14:665-9 [1966]); and Keogh et al., Appl. Microbiol., 19:928-31 [1970]). In some embodiments, the starter culture is modified to contain the sources of formate and purine, as discussed above.

In some embodiments, while the concentrations of the various components of a subject starter composition varies, the lactic acid bacteria are present at a concentration in the range of about 10⁶ CFU/g to about 10¹³ CFU/g, for example. In some further embodiments, the sources of formate and purine are each independently present at a concentration of about 5% to about 80% (e.g., about 10% to about 50% (w/w)). In still further embodiments, the enzyme is present at a concentration of about 10 mg/ml to about 200 mg/ml. In still further embodiments, the concentration of lactase blended within the starter composition is set to provide a lactase concentration in the process milk from about 0.5 to about 20 units of lactase per milliliter of milk.

In one exemplary embodiment, a starter composition is made by adding appropriate amounts of the powdered forms of the sources of formate and purine to a lyophilized (i.e., freeze dried) cell culture. In other embodiments, the starter composition is made by blending a frozen bacterial pellet with a frozen solution containing the sources of formate and purine. In embodiments containing lactase, the lactase is added at any time during the preparation of the starter composition.

In some embodiments, the starter culture is made by inoculating hydrolyzed 1-5% milk supplemented with carbohydrates (e.g., glucose, lactose, and/or sucrose), yeast extract, peptones and/or minerals. During the fermentation, the pH is controlled, such that it is maintained at about pH 6 (i.e., using NH₄OH). In some embodiments, when the base consumption stops, the fermentate is cooled and the cells concentrated in a centrifuge. In some embodiments, the concentrate is then canned or placed in suitable containers for freezing in liquid nitrogen or dripped as drops into liquid nitrogen. In some other embodiments, the pellets containing the sources of formate and purine are also made by dripping solutions containing those compounds (either separately or mixed) into liquid nitrogen. In some embodiments, the culture pellets and formate/purine pellets are then mixed to provide starter compositions that contain those components suitable concentrations. In some embodiments, the starter composition is combined with a dairy substrate to produce the bacterial cultures described above.

### Kits

The present invention also provides kits for inoculating a dairy substrate with a starter culture. In some preferred embodiments, the kits comprise at least one culture of lactic acid bacteria (frozen or liquid), isolated lactase enzyme (powdered or a liquid stabilized in glycerol), a source of formate and a source of purine (powdered or a dissolved in a liquid, such as water). In some embodiments, the various components of the kit are present in separate containers, while in other embodiments, compatible components are precombined into a single container, as desired. In some particularly preferred embodiments, the kits further comprise a starter composition.

In some embodiments, the present invention provides kits for inoculating a dairy substrate. In some embodiments, the kits comprise a frozen or liquid starter culture of lactic acid bacteria, at least one source of formate and/or at least one source of purine. In some embodiments, the purine and/or formate sources are in powder form, while in other embodiments the purine and/or formate sources are in liquid (e.g., dissolved in water). In some embodiments, the various components of the kit are provided in separate containers, while in some further embodiments, compatible components are precombined into a single container, as desired. In some preferred embodiments, the kits contain at least one starter composition.

In addition to above-mentioned components, in additional embodiments, the kits further comprise instructions for using the components of the kit to practice the methods of the present invention. The instructions for practicing the subject methods are generally recorded on a suitable recording medium, and may contain information on the amount of dairy substrate that is to be inoculated. For example, in some embodiments, the instructions are printed on a substrate, such as paper or plastic, etc. As such, in some embodiments, the instructions are provided in the kits as a package insert and/or on the labeling of the kit or components thereof (i.e., associated with the packaging or subpackaging) etc.

### Other Formats

In some further embodiments of the present invention, the fermented milk product is packaged (e.g., to make a product that can be shipped and sold). In other embodiments, the fermented milk product is shipped to a remote location prior to packaging. In some embodiments, the fermented milk product is sold as a drink, while in other embodiments, it is sold as a food (e.g., cheese, fermented milk drink, lactic acid bacteria beverage, yogurt, cultured milk, kefir, or the like). In some embodiments, the product is plain, while in other embodiments, it is flavored, sweetened, and/or contain fruit or vegetables. In some embodiments, it is a soft product, while in other embodiments, it is hard, liquid, or frozen.

In order to further illustrate the present invention and advantages thereof, the following specific examples are given with the understanding that they are being offered to illustrate the present invention and should not be construed in any way as limiting its scope.

### EXPERIMENTAL

In the experimental disclosure which follows, the following abbreviations apply: ° C. (degrees Centigrade); H₂O (water); gm (grams); μg and ug (micrograms); mg (milligrams); ng (nanograms); μl and ul (microliters); ml (milliliters); mm (millimeters); nm (nanometers); μm and um (micrometer); M (molar); mM (millimolar); μM and uM (micromolar); U (units); sec (seconds); min(s) (minute/minutes); hr(s) (hour/hours); sd and SD (standard deviation); PBS (phosphate buffered saline [150 mM NaCl, 10 mM sodium phosphate buffer, pH 7.2]); w/v (weight to volume); v/v (volume to volume); CFU (colony forming units); NCFM (North Carolina Food Microbiology Department); Becton Dickinson (Becton Dickinson Diagnostic Systems, Sparks, Md.); (Difco Laboratories, Detroit, Mich.); GIBCO BRL or Gibco BRL (Life Technologies, Inc., Gaithersburg, Md.); UNC (University of North Carolina); and ATCC (American Type Culture Collection, Manassas, Va.).

### Example 1

### Growth of Lactococcus with Formate and Purine Supplementation

In these experiments, the growth of Lactococcus in the presence of supplemented formate and purine was investigated. Equal amounts of milk (pasteurized milk w/1% fat) were added to four 1 liter Pyrex bottles. The bottles were placed in the water bath at 32° C.

All bottles were inoculated with a mesophilic culture of Lactococcus (0.022%). When the milk reached 32° C., three of the four bottles were supplemented with Na formate and/or disodium-5′-inosinate (IMP), as shown below.

| Bottle | Na-Formate | IMP |
|---|---|---|
| 1 | 0 | 0 |
| 2 | 5 ppm | 0 |
| 3 | 0 | 10 ppm |
| 4 | 5 ppm | 10 ppm |

The initial pH of the milk was recorded. The pH in the bottles was measured every hour for the first two hours and then every half hour until a pH of around 4.6 was reached.

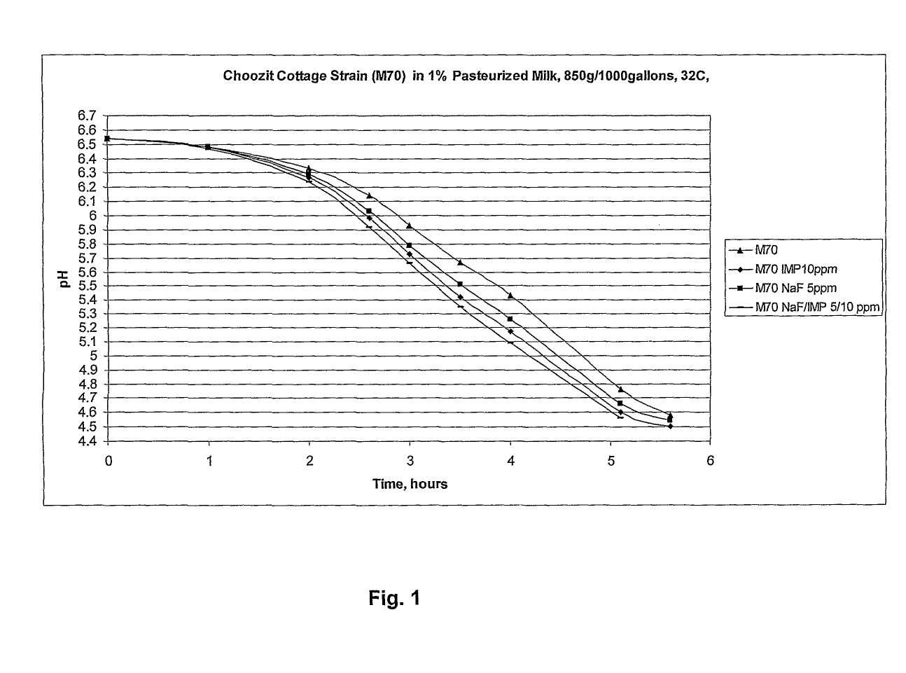

The pH of each of the bottles, as a function of time, is shown below, and graphed in FIG. 1.

| | | | M70 | M70 |
|---|---|---|---|---|
| Time | | M70 IMP | NaF | NaF/IMP |
| (Hours) | M70 | 10 ppm | 5 ppm | 5/10 ppm |
| 0 | 6.54 | 6.54 | 6.54 | 6.54 |
| 1 | 6.48 | 6.48 | 6.48 | 6.47 |
| 2 | 6.33 | 6.27 | 6.29 | 6.24 |
| 2.6 | 6.14 | 5.98 | 6.03 | 5.92 |
| 3 | 5.93 | 5.73 | 5.79 | 5.66 |
| 3.5 | 5.67 | 5.42 | 5.51 | 5.351 |
| 4 | 5.43 | 5.17 | 5.26 | 5.09 |
| 5.1 | 4.76 | 4.6 | 4.66 | 4.56 |
| 5.6 | 4.58 | 4.5 | 4.54 | |

### Example 2

### Growth of Lactococcus in Lactase-Supplemented Media

Milk was equilibrated to 86° F. (30° C.) and inoculated with a mesophilic culture of Lactococcus. The culture used was a concentrated culture. The inoculation rate was 0.025%. Lactase was added in accordance with the following table, and the pH of the culture was measured 4.5 hrs later.

| | Lactase | pH at 4.5 hours |
|---|---|---|
| 1 (Control) | 0 | 5.33 |
| 2 | 0.5 u/ml of milk | 5.18 |
| 3 | 1.0 | 5.16 |
| 4 | 2.0 | 5.12 |
| 5 | 5.0 | 5.05 |
| 6 | 10.0 | 5.00 |

After the fermentation to pH 4.6 the milk was cooled and stored a 45° F.

### Example 3

### Growth of Freeze-Dried Yogurt Culture with Lactase Supplementation

The same experiment as described in Example 2 was performed w/ a freeze dried yogurt culture (Streptococcus thermophilus and Lactobacillus bulgaricus). In this example the milk was at a temperature of 108° F. Inoculation rate was 0.003%. Again, lactase was added in accordance with the following table, and the pH of the culture was measured 2.5 hrs later.

| Sample | | Lactase | pH at 2.5 hours |
|---|---|---|---|
| Bottle | 1 (Control) | 0 | 5.75 |
| | 2 | 0.5 | 5.69 |
| | 3 | 1.0 | 5.49 |
| | 4 | 2.0 | 5.46 |
| | 5 | 5.0 | 5.44 |

After the fermentation to pH 4.6 the samples were cooled to 45 F and stored.

### Example 4

### Growth in Formate, Purine and Lactase-Supplemented Medium

In these experiments, the combination booster (i.e., Na-formate, IMP, and lactase) were tested in order to determine whether this would provide a beneficial effect. In this case a mesophilic strain was used. In this Example the milk was at a temperature of 86° F. (30° C.). The inoculation rate of the culture was 0.025%, and the pH was measured at 4 hours. The results are provided below.

| | Lactase | ppm NaF | ppm IMP | pH at 4 hours |
|---|---|---|---|---|
| Control | 0 | 0 | 0 | 5.45 |
| 1. | 0 | 5 | 10 | 5.18 |
| 2. | 5 u/ml | 5 | 10 | 4.98 |
| 3. | 5 u/ml | 0 | 0 | 5.28 |

What is claimed is:

1. A bacterial culture comprising a dairy substrate, a population of lactic acid bacteria, exogenous lactase, and/or a source of formate, and/or a source of purine.

2. The bacterial culture of claim 1 comprising a dairy substrate, a population of lactic acid bacteria, an exogenous lactase, a source of formate, and a source of purine

3. The bacterial culture of claim 1, wherein the exogenous lactase is at a concentration of about 0.5 to about 20 units of lactase per milliliter of said bacterial culture.

4. The bacterial culture of claim 1, wherein said dairy substrate is milk or cream.

5. The bacterial culture of claim 1, wherein said population of bacteria is a population of bacteria of the genus Lactococcus, Lactobacillus and/or Streptococcus.

6. The bacterial culture of claim 1, wherein the source of formate is sodium formate.

7. The bacterial culture of claim 1, wherein the source of formate is present at a concentration of about 0.25 ppm to about 50 ppm.

8. The bacterial culture of claim 1, wherein the source of purine is inositol 5′ monophosphate, inosine, hypoxantine and/or guanine.

9. The bacterial culture of claim 1, wherein said source of purine is present at a concentration of about 0.5 ppm to about 100 ppm.

10. The bacterial culture of claim 1, wherein said population of lactic acid bacteria are at lag phase.

11. The bacterial culture of claim 1, wherein said bacterial culture is made by inoculating a dairy substrate with a starter composition comprising:
a) a population of lactic acid bacteria at a concentration in the range of about 10⁶ to about 10¹³ CFU/g;
b) an exogenous lactase at a concentration sufficient to provide a lactase concentration in the range of about 0.5 to about 20 units of lactase per milliliter in said bacterial culture, and a source of formate at a concentration of about 10% to about 50% (w/w).

12. The bacterial culture of claim 11, further comprising a source of purine at a concentration sufficient to provide a concentration of about 10% to about 50% (w/w) in said bacterial culture.

13. A method comprising maintaining the bacterial culture of claim 1, under conditions suitable for fermentation, to produce a fermented milk product.

14. The method of claim 13, wherein said method provides for the production of lactic acid by said population of lactic acid bacteria.

15. The method of claim 13, wherein said fermented milk product is yogurt, cheese, crème fraiche or fermented milk beverage.

16. The method of claim 13, further comprising adding a flavoring to said fermented milk product.

17. The method of claim 13, further comprising adding a stabilizer to said fermented milk product.

18. The method of claim 13, further comprising packaging said fermented milk product.

19. A fermented product made by the method of claim 13.

20. A composition for starting a culture, comprising:
a) a population of lactic acid bacteria at a concentration in the range of about 10⁶ to about 10¹³ CFU/g;
b) an exogenous lactase provided in a concentration sufficient to provide a lactase concentration in the range of about 0.5 to about 20 units of lactase per milliliter in said composition, and a source of formate at a concentration of about 10% to about 50% (w/w);

21. The composition of claim 20, further comprising a source of purine sufficient to provide a concentration of about 10% to about 50% (w/w) purine in said composition

22. The composition of claim 20, wherein said composition is a frozen composition made by blending a frozen bacterial pellet with a frozen solution containing said source of formate and said source of purine.

23. The frozen composition of claim 20, wherein said composition is made by adding a powdered source of formate and a powdered source of purine to a lyophilized population of lactic acid bacteria.

24. A method comprising combining the frozen composition of claim 20, with a dairy substrate to make a bacterial culture.
